(19) United States
(12) Patent Application Publication
(10) Pub. No.: US 2026/0363763 A1
(43) Pub. Date: Nov. 19, 2026

(54) BASIVERTEBRAL NERVE ABLATION PROBE

(71) Applicant: Baylis Medical Technologies Inc., Mississauga (CA)

(72) Inventors: Neil Godara, Milton (CA); Christopher Breadner, Ajax (CA); Emily Ma, Windsor (CA)

(21) Appl. No.: 19/677,290

(22) Filed: May 14, 2026

Related U.S. Application Data
(60) Provisional application No. 63/806,795, filed on May 16, 2025.

(51) Int. Cl. A61B 18/14; A61B 18/00

(52) U.S. Cl. CPC A61B 18/1492; A61B 2018/00023; A61B 2018/0016; A61B 2018/00339; A61B 2018/00434; A61B 2018/00565; A61B 2018/00577; A61B 2018/00791; A61B 2018/1427

(57) ABSTRACT

A system and method for channeling a path into bone and delivering radiofrequency energy are disclosed. The systems includes an introducer, a curved sheath, and a radiofrequency probe having a sharp tip capable of channeling through bone. The sharp tip has a diameter larger than the diameter of the probe sharp. In some configurations, the probe has an asymmetric shape.

### TECHNICAL FIELD

The disclosure relates to systems and methods for ablating tissue, and more specifically to creating a channel and ablating a nerve within a bone.

### BACKGROUND OF THE ART

Current basivertebral nerve (BVN) ablation systems use a combination of devices to gain access to the vertebra. This is required because the normal access to the vertebra is through the back and entering the pedicle of the vertebra. The pedicle offers a safe path past the spinal canal, as any device would be fully inside bone, and therefore avoids inadvertent damage to neural structures.

After pedicle access is achieved, the requirement is to place the probe parallel to the posterior wall of the vertebral body in a position that is within the most posterior half of the vertebral body. The reason why this is required is because the BVN forms a trunk in the posterior half of the vertebral before branching out and enervating the endplates.

Typically, this is achieved by introducing four separate components:
1) A bone introducer to gain access to the pedicle. This is typically advanced until the tip is in line with the posterior wall of the vertebral body.
2) A curved sheath that is inserted into the bone introducer. The curved sheath acts to change the axis of the primary lumen, such that it is no mostly pointed in a direction that is parallel to the posterior wall.
3) A straight stylet this is inserted into the curved sheath and extends into the bony channel of the vertebra. This stylet is use to create a channel or path in the bone and is then removed from the curved sheath.
4) A flexible probe that is inserted into the curved sheath and is placed inside the channel created by the straight style.

Such a system is problematic as it comprises multiple parts, reducing the efficiency of the procedure. Additionally, having a straight stylet create a path, and having a probe that is not capable of creating a path itself, results in a poor probe-bone interface as there will necessarily be a gap between the probe, and therefore any electrodes on the probe, and the bony tissue. This results in poor ablation efficacy. There is therefore a need for a more efficient procedure, requiring fewer devices/exchanges and/or improving probe-bone contact.

### BRIEF DESCRIPTION OF THE DRAWINGS

In order that the invention may be readily understood, embodiments of the invention are illustrated by way of examples in the accompanying drawings, in which:

FIG. 1 is an illustration of medical devices creating a first path within a bone;

FIG. 2 is an illustration of a sharp tipped probe forming a channel towards a treatment location, in accordance with an embodiment of the present invention;

FIG. 3 is an illustration of devices that may be used to facilitate creating a channel towards a treatment location within a bone, in accordance with an embodiment of the present invention;

FIG. 4 is an illustration of a radiofrequency probe having a sharp distal tip, in accordance with an embodiment of the present invention; and

FIGS. 5A to 5H are illustrations of various distal tip shapes in accordance with several embodiments of the present invention.

In the drawings, like reference characters generally refer to the same parts throughout the different views. Also, the drawings are not necessarily to scale, emphasis instead being placed upon generally illustrating the various concepts discussed herein.

### DETAILED DESCRIPTION

The inventors have developed a novel systems and methods for creating a channel within the bone and delivering energy. One embodiment of the present invention comprises the following components:
1) A bone introducer to gain access to the pedicle. This is typically advanced until the tip is in line with the posterior wall of the vertebral body.
2) A curved sheath that is inserted into the bone introducer. The curved sheath acts to change the axis of the primary lumen, such that it is pointed in a direction that is parallel to the posterior wall.
3) A coaxial, bipolar, radiofrequency ablation probe that is inserted into the curved sheath. The probe is sufficiently flexible to navigate the curve of the sheath and has the necessary axial stiffness such that it can be advanced into bone by the user directly or with the aid of a surgical mallet. The probe comprises a sharp tip that is capable of being inserted into bone.

In one embodiment of the present invention, a system for channeling a path into bone and delivering radiofrequency energy is provided, the system comprising an introducer sheath having a central lumen extending between a proximal opening and a distal opening; and a radiofrequency ablation probe, the radiofrequency ablation probe comprising: an elongate member having a sharp distal tip; and at least one electrode for delivering radiofrequency energy, the at least one electrode located in a distal region of the elongate member; wherein at least a portion of the sharp distal tip defines a tip diameter greater than a diameter of at least a portion of the elongate member.

In a specific embodiment, the sharp distal tip defines a tapered portion.

In another embodiment, the sharp distal tip defines a stepped portion.

In yet another embodiment, the sharp distal tip defines an asymmetric shape.

In a further embodiment, the probe comprises a temperature sensor.

In a yet further further embodiment, at least a portion of the elongate member defines a lumen operable to circulate a cooling fluid therein.

In a particular embodiment, the system further comprises a curved sheath sized and configured to be received within the central lumen of the introducer sheath and operable to modify a direction of a longitudinal axis of the introducer sheath when inserted therethrough to apply a curve to the longitudinal axis. Together, the curved sheath and introducer sheath form a curved sheath assembly.

In some such embodiments, the curved sheath defines a lumen and wherein the radiofrequency ablation probe is sized to be insertable into the curved sheath lumen. More specifically, the probe may be sufficiently flexible to follow the curve of the sheath lumen.

In another broad aspect of the present invention, the invention comprises a probe for creating a channel through tissue and for delivering energy within a patient's body. In such aspects, the probe may comprise an elongate member; at least one electrode for delivering energy to a treatment location; and a sharp distal tip, the sharp distal tip defining a tip diameter; wherein the tip diameter is greater than a diameter of at least a portion of the elongate member.

In an embodiment of this aspect, the probe may comprise a temperature sensor and at least a portion of the elongate member may define a lumen operable to circulate a cooling fluid therein.

In a further broad aspect of the present invention, a method of delivering energy to a bony tissue is provided, the method comprising the steps of: advancing a curved sheath assembly through a pedicle into a vertebral body; inserting an ablation probe through the curved sheath assembly; advancing the ablation probe through a distal opening of the curved sheath assembly, such that the ablation probe exits the distal opening in a straight orientation; and advancing the ablation probe through bony tissue using a distal tip of the ablation probe which is capable of penetrating bone independently.

In some specific embodiments, the probe is capable of creating its own path through bone without the need of a stylet or other device to create a path for its entry. Additionally, the probe has both the radial flexibility and axial stiffness to aid in both insertion into the sheath and entry to the bone.

As the probe is nominally straight, it generally maintains the direction in which it is inserted. As such, when coupled with a curved sheath that directs the probe in a path that is parallel to the posterior wall of the vertebral body, the probe will generally track in this direction.

In some embodiments, the sharp tip of the probe is asymmetrical, such that one face of the tip works to direct the probe in a certain direction. For example, once the straight probe is placed in the curved sheath, the radial flexibility of the probe will still impart some force on the sheath, such that the combination of the two will act to reduce the curve of the sheath. This will be accentuated the further the probe is extended into the bony tissue. To counteract this, a beveled face at the tip of the probe, pointing anteriorly, will act to push the probe tip posteriorly. This would counteract the forces that are otherwise pushing the probe posteriorly.

As the device that is creating the channel is also capable of delivering and measuring electrical signals, it is possible for this invention to confirm the location of the nerve using sensory stimulation. For example, the probe could deliver bipolar stimulation outputs such that the patient can confirm that the pain being evoked is concordant with their pain symptoms. In this way, the probe can be optimally placed, and unnecessary bone access is avoided, as would be the case if a stylet was placed to create a channel without knowing the optimal position of the probe.

According to another embodiment of the present invention, a basivertebral nerve (BVN) ablation probe is disclosed that features an integrated self-advancing tip capable of channeling into cancellous bone without the aid of a separate stylet, trocar, or drilling instrument. Unlike prior art systems that rely on sequential use of a curved stylet and cannula to create the bone path or require the probe to bend or deflect upon exiting a straight introducer, the probe of the present invention emerges straight from a pre-curved sheath, maintaining a consistent axial orientation.

The probe of the present invention enables i) streamlined procedural workflow, eliminating instrument exchanges, ii) improved targeting by preserving orientation during sheath exit, and iii) mechanical channeling into vertebral bone via the probe tip itself, reducing the need for additional bone access tools.

The present invention differs from prior art system that require a separate curved stylet or cannula to define the access trajectory, or use steerable or shape-memory elements to achieve directional control post-exit.

In one embodiment, the invention provides a bone-penetrating ablation probe that: i) exits straight from a pre-curved sheath, maintaining linear alignment with the sheath axis; ii) features a self-advancing tip capable of penetrating cancellous bone directly, eliminating the need for a separate introducer, trocar, or curved stylet; iii) simplifies the procedural workflow by combining bone access and ablation in a single device; and iv) enables precise lesion placement at or near the basivertebral nerve without requiring probe steering or post-deployment deflection.

The system is specifically designed for compatibility with curved sheaths that establish the access trajectory through the pedicle and into the vertebral body, thereby preserving targeting accuracy while streamlining tool exchanges.

In some embodiments, the distal tip of the probe includes a raised or enlarged portion—either at the terminal end or slightly proximal to it—having a diameter greater than that of the probe shaft. This configuration allows the probe to: i) create a channel in cancellous bone with a slightly larger diameter than the shaft, ii) minimize contact friction between the probe body and bone during insertion and withdrawal, iii) facilitate easier advancement into the vertebral body by reducing drag, and iv) enable smoother removal, as only the enlarged region contacts the channel walls with appreciable force.

This geometry may be implemented as a gradual taper, a bulbous tip, a stepped shoulder, or an abrasive or textured ring. The probe shaft trailing the enlarged region is undersized relative to the channel it enters, thereby reducing binding or resistance during use.

In some embodiments, the distal face of the probe tip is asymmetric, for example, angled or beveled, to enable passive directional bias during advancement. This asymmetry preferentially deflects the probe posteriorly, guiding it toward the basivertebral nerve target location within the vertebral body.

This directional feature, enhances targeting precision without requiring active steering mechanisms, leverages the natural interaction between the probe tip and cancellous bone to guide trajectory, and can be combined with the raised diameter section to balance channeling efficiency and directional control. The angle and geometry of the tip may be fixed or variable (e.g., replaceable or formed by controlled abrasion). The asymmetry may be planar, helical, or offset from the probe's central axis.

### System and Radiofrequency Probe

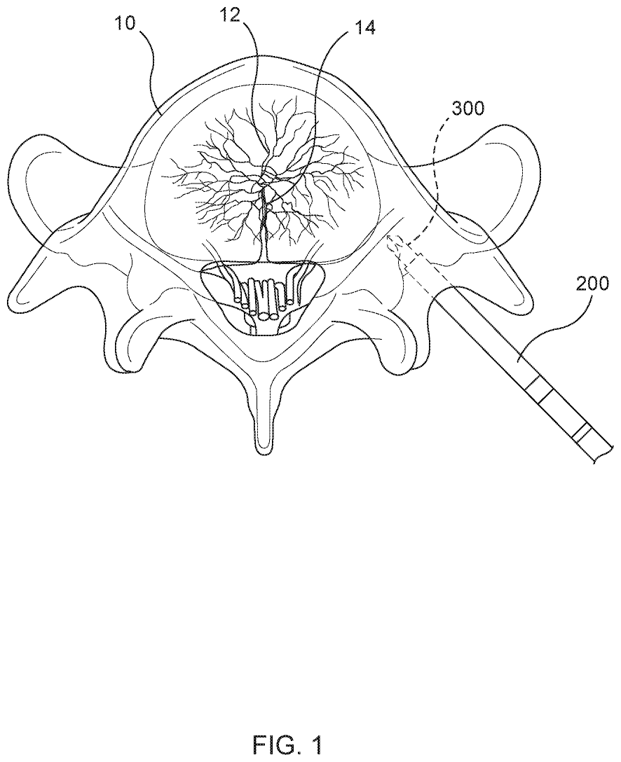

With reference to FIG. 1, according to one embodiment of the present invention, the first step in ablating a BVN nerve is to gain access to bony tissue 10. The basivertebral nerve 12 has a trunk 14, and a channel must be made across or near the trunk 14 in order to ablate the nerve. In some cases, a bone introducer 200 and a straight stylet 300 are used to pierce the pedicle.

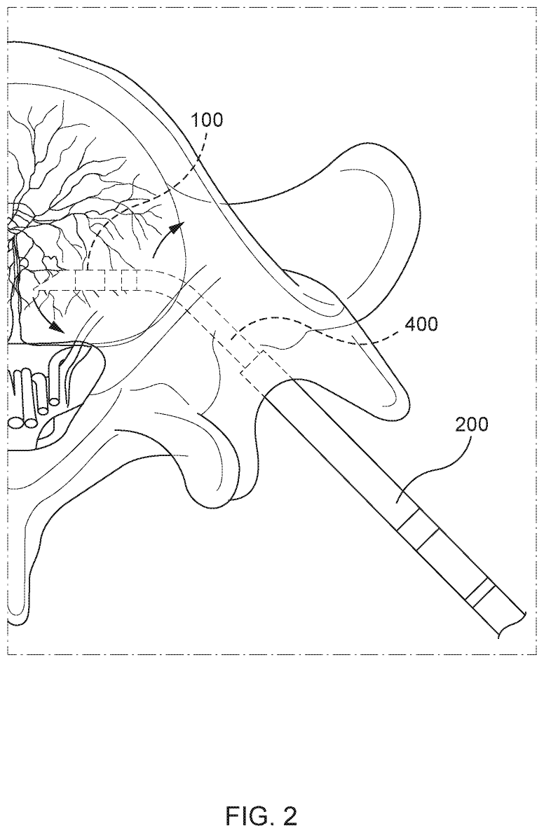

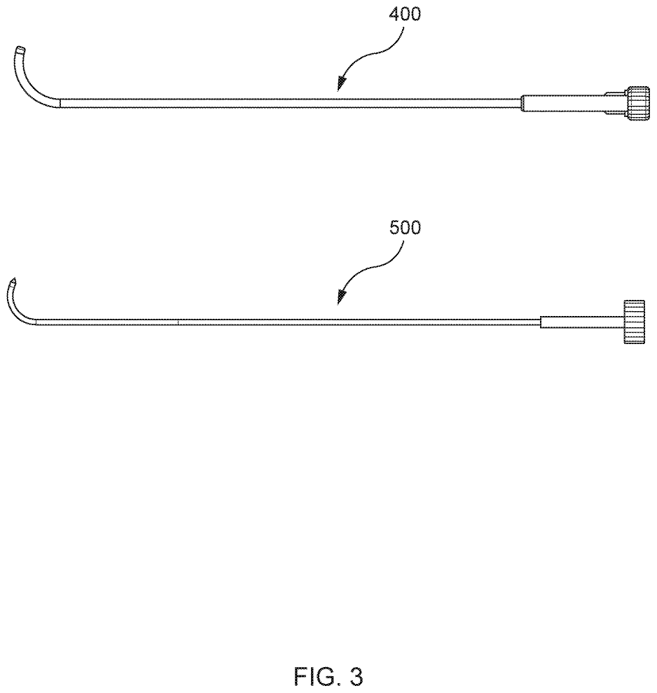

With reference to FIG. 2, in one embodiment, once a straight channel has been used to gain access to a region within the bone, a probe 100 having a sharp tip is used to create a channel the rest of the way to the treatment location (for example, trunk 14). In some embodiments, a flexible, curved sheath 400 is used to aim the probe 400 and create a curved channel to advance the probe 100 to the treatment location. The curved sheath 400 acts to change the axis of the primary or central lumen of the introducer sheath so that it is parallel to a posterior wall. In some embodiments, a curved stylet 500 is used to create a portion of the channel to reach the treatment site. Curved sheath 400 and curved stylet 500 are shown in FIG. 3. The combination of an introducer sheath and or more of a curved sheath or curved stylet may be referred to as a curved sheath assembly.

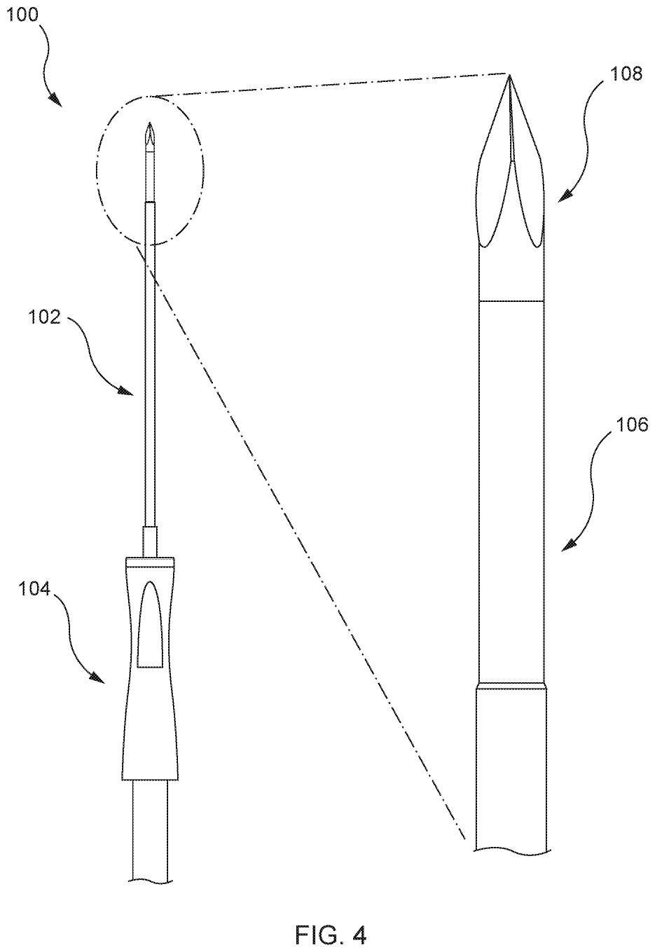

With reference to FIG. 4, in one embodiment, radiofrequency ablation probe 100 (also referred to as “radiofrequency probe” or “RF probe”) comprises an elongate member 102 and a handle 104. A proximal end of the elongate member 102 is coupled to the handle 104. Handle 104 comprises various wires to connect to an energy source, for example a generator, for providing energy to ablate a nerve. RF probe 100 comprises one or more electrodes 106. In one example, RF probe 100 is a bipolar probe and comprises two electrodes 106a and 106b (not shown). In one embodiment, probe 100 comprises a strike plate (not shown) on the handle 104, so that the probe 100 may be hammered into bone. At a distal end of elongate member 102 is a sharp distal tip 108.

In one embodiment, RF probe 100 comprises a sharp distal tip 108 that is enlarged, in other words, larger than the diameter of the elongate member 102. Sharp distal tip 108 may have various shapes, as described herein.

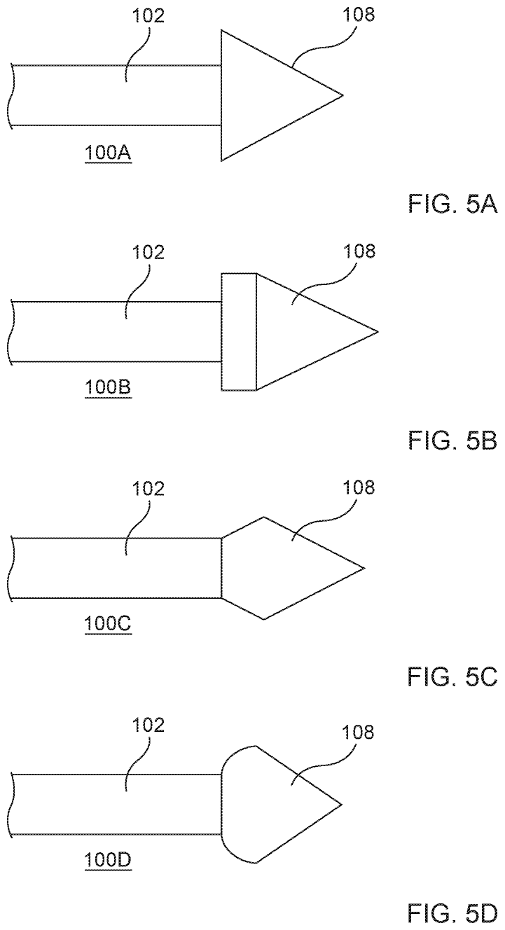

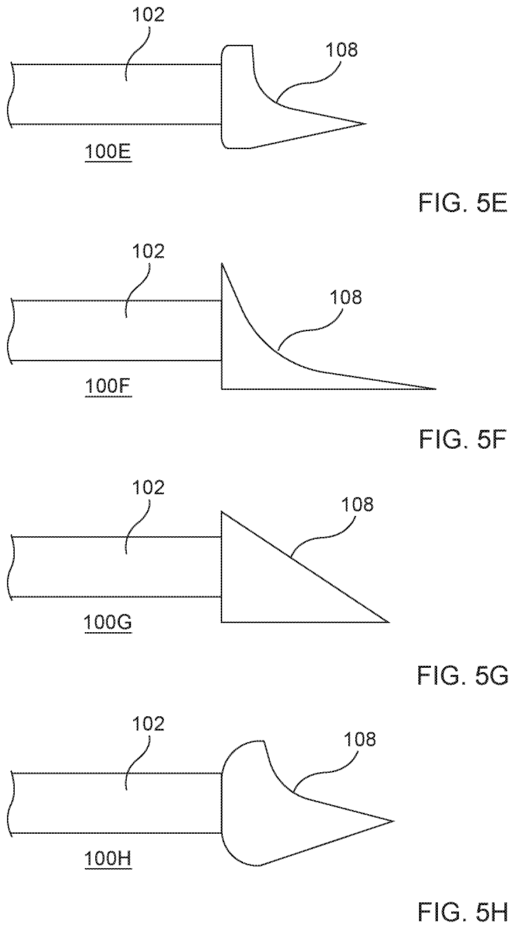

With reference to FIGS. 5A to 5H, in one embodiment, sharp distal tip 108 has an overall outer diameter that is greater than the diameter of elongate member 102. The geometry of the tip may have a taper, a bulbous tip, a step, a shoulder, an abrasive ring, a textured ring or any other shape where overall size of the tip is greater than the diameter of the elongate member 102. Example tip shapes are shown in FIGS. 5A to 5H.

The enlarged tip may provide advantages during use, making it easier to remove the probe 100 after it has been used to create a channel within the bone.

In some embodiments, for example probe 100E, 100F, 100G, and 100H, the sharp distal tip 108 has an asymmetric shape.

In other embodiments, the tip 108 has a shape similar to any shape shown in FIGS. 5A to 5H, but the diameter is the same as the diameter of the elongate member.

In some embodiments, probe 100 is a cooled probe that uses a cooling fluid to cool a portion of the elongate member.

The devices described herein may be used as part of a method for creating a channel within a vertebral body for example for a basivertebral nerve ablation, the method comprising the steps of: advancing a pre-curved introducer sheath through a pedicle into the vertebral body; and inserting a self-channeling ablation probe through the sheath, wherein the probe exits in a straight configuration and includes a distal tip capable of penetrating cancellous bone without a separate bone-cutting or steering instrument.

In another embodiment, the devices may be used for a method of reducing probe friction during insertion into cancellous bone, the method comprising the steps of: inserting an ablation probe into bone; wherein the distal portion of the probe has a diameter greater than that of its shaft, such that the created bone channel has a larger diameter than the shaft, thereby limiting contact friction between the probe body and the bone.

In another embodiment a method of guiding a probe within a vertebral body toward a target ablation site is disclosed, the method comprising the steps of: inserting a probe having a distal tip with an angled or asymmetric face, where the asymmetry causes the tip to passively deflect toward a posterior direction during advancement through cancellous bone.

In some embodiments, the probe is configured for radiofrequency ablation and includes one or more electrodes adjacent the distal tip.

In some embodiments, the raised diameter portion is bulbous, tapered, or stepped.

In some embodiments, the directional bias aligns the tip with the basivertebral nerve target zone.

The embodiment(s) of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention.

1. A system for channeling a path into bone and delivering radiofrequency energy, the system comprising:
an introducer sheath having a central lumen extending between a proximal opening and a distal opening; and
a radiofrequency ablation probe, the radiofrequency ablation probe comprising:
an elongate member having a sharp distal tip; and
at least one electrode for delivering radiofrequency energy, the at least one electrode located in a distal region of the elongate member;
wherein at least a portion of the sharp distal tip defines a tip diameter greater than a diameter of at least a portion of the elongate member.

2. The system of claim 1, wherein the sharp distal tip defines a tapered portion.

3. The system of claim 1, wherein the sharp distal tip defines a stepped portion.

4. The system of claim 1, wherein the sharp distal tip defines an asymmetric shape.

5. The system of claim 1, wherein the radiofrequency probe comprises a temperature sensor.

6. The system of claim 1, wherein at least a portion of the elongate member defines a lumen operable to circulate a cooling fluid therein.

7. The system of claim 1, further comprising a curved sheath sized and configured to be received within the central lumen of the introducer sheath and operable to modify a direction of a longitudinal axis of the introducer sheath when inserted therethrough to apply a curve to the longitudinal axis.

8. The system of claim 7, wherein the curved sheath defines a lumen and wherein the radiofrequency ablation probe is sized to be insertable into the curved sheath lumen.

9. The system of claim 8, wherein the radiofrequency ablation probe is sufficiently flexible to follow the curve.

10. The system of claim 1, the at least one electrode comprising two electrodes operable in a bipolar manner.

11. A probe for creating a channel through tissue and for delivering energy within a patient's body, the probe comprising:
an elongate member;
at least one electrode for delivering energy to a treatment location; and
a sharp distal tip, the sharp distal tip defining a tip diameter;
wherein the tip diameter is greater than a diameter of at least a portion of the elongate member.

12. The probe of claim 11, further comprising a temperature sensor.

13. The probe of claim 11, wherein at least a portion of the elongate member defines a lumen operable to circulate a cooling fluid therein.

14. A method of delivering energy to a bony tissue, the method comprising the steps of:
advancing a curved sheath assembly through a pedicle into a vertebral body;
inserting an ablation probe through the curved sheath assembly;
advancing the ablation probe through a distal opening of the curved sheath assembly, such that the ablation probe exits the distal opening in a straight orientation; and
advancing the ablation probe through bony tissue using a distal tip of the ablation probe which is capable of penetrating bone independently.
